(19) United States
(12) Patent Application Publication
(10) Pub. No.: US 2010/0267653 A1
(43) Pub. Date: Oct. 21, 2010

(54) USE OF COUMARIN DERIVATIVES IN ANTIFUNGAL THERAPY

(76) Inventor: Colin Stewart, Craibstone (GB)

Correspondence Address: MORGAN, LEWIS & BOCKIUS LLP (SF), One Market, Spear Street Tower, Suite 2800, San Francisco, CA, 94105, US

(21) Appl. No.: 12/303,958

(22) PCT Filed: Jun. 5, 2007
(86) PCT No.: PCT/GB07/02062

§ 371 Date: Jun. 30, 2010

Related U.S. Application Data
(60) Provisional application No. 60/821,228, filed on Aug. 2, 2006.

(30) Foreign Application Priority Data
Jun. 6, 2006 (GB) 0611115.7

(51) Int. Cl. A61K 31/7034; C07D 311/74; C07H 15/203; C07D 311/10; A61K 31/352; A61P 31/10

(52) U.S. Cl. 514/27; 549/290; 536/18.1; 549/283; 514/457

(57) ABSTRACT

The invention provides coumarin compounds, in particular glycosidic coumarin compounds, useful in the treatment of dermatophyte fungal infections.

### FIELD OF THE INVENTION

This invention relates to compounds and their use in antifungal therapy.

### BACKGROUND TO THE INVENTION

Dermatophytes are fungi that are dermal pathogens. The dermatophytes comprise fungi of the genera Trichophyton, Epidermophyton and Microsporum. A common dermatophytic fungal infection is onychomycosis, which refers to an infection of the nail, which can cause the nail to split, flake and thicken. Onychomycosis is most commonly caused by Trichophyton rubrum, but other dermatophytes such as Epidermophyton floccosum, Microsporum canis and Trichophyton interdigitale may also be causative.

Coumarins are compounds which comprise a chromenone ring, often a chromen-2-one or chromen-4-one ring. The compounds occur naturally and can also be synthesised, for example by a Perkin or Pechman synthesis. The structure of coumarin itself is shown below:

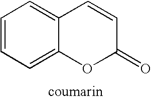

Selected coumarins are known to have antifungal activity. For example, Sardari et al (Bioorg. Med. Chem. 7, 1999, 1933-1940) describe how a limited number of coumarins are active against Candida albicans, Cryptococcus neoformans, Saccharomyces cerevisiae and Aspergillus niger. Among the coumarins tested were esculetin and esculin (a glycosidic form of esculetin), which were found to have minimal antifungal activity against the fungi tested.

Apart from warfarin, the use of coumarins in clinical settings has been minimal. However, an example of a coumarin which has been used in the clinic is esculin, which is used as a component of a medicament (Proctosedyl®) for treating haemorrhoids and rectal lesions.

Despite their limited known potential as antifungals, the insolubility of coumarins at the active range of concentrations, on the whole, combined with potential safety and toxicity issues may rule out any practical direct therapeutic application of this class of compounds.

### SUMMARY OF THE INVENTION

The present invention is based, at least in part, on the discovery that glycosidic coumarin compounds such as esculin are effective in the treatment of onychomycosis and other dermatophytic infections. Without wishing to be bound by theory, it is believed that dermatophytes and other microbes with which they may be present on the skin (commensal or pathogenic fungi or bacteria) can metabolise glycosidic groups attached to coumarin compounds, converting the compounds into the active core coumarin which is active against the fungi. For example, it has been demonstrated that dermatophytes produce the enzyme β-glucosidase (this application; K Otsuka, et al., (1979), Chem Pharm Bull, 27:2042-2047) that could remove the sugar group of esculin and convert it into esculetin, which is then antifungal. Thus, the sugar group may, in a sense, be considered to be a pro-drug functionality. The use of glycosidic coumarins as medicaments may be desirable over non-glycosidic forms, because the former are more soluble and therefore can be practically incorporated into suitable vehicles for therapeutic application and are also considered to be safer for handling and application.

The present invention is also based on the surprising finding that, despite only being soluble at low concentrations, halogenated coumarin compounds for example 7-hydroxy-4-(trifluoromethyl)coumarin have significant antifungal activity.

Accordingly, a first aspect of the invention is a coumarin compound or a pharmaceutically acceptable salt or pro-drug thereof, for use in the treatment, prevention or delay of progression of a dermatophytic infection in a patient. The coumarin compound may be a glycosidic coumarin compound. Alternatively the coumarin compound may be a halogenated coumarin compound.

A second aspect of the invention is a pharmaceutical formulation comprising a coumarin compound, for example a glycosidic coumarin compound, or a pharmaceutically acceptable salt or prodrug thereof, for use in the treatment, prevention or delay of progression of a dermatophytic infection in a patient.

Another aspect of the invention concerns the use of a coumarin compound, for example a glycosidic coumarin compound, or a pharmaceutically acceptable salt or prodrug thereof, for the manufacture of a medicament for the treatment, prevention or delay of progression of a fungal infection, such as a dermatophytic infection.

The invention further provides a method for the treatment, prevention or delay of progression of a dermatophytic infection, which comprises administering to a patient a therapeutically effective amount of a coumarin compound, for example a glycosidic coumarin compound, or a pharmaceutically acceptable salt or prodrug thereof.

Compounds of the invention may exist in different forms, such as free acids, free bases, esters and other prodrugs, salts and tautomers, for example, and the disclosure includes all variant forms of these compounds.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Features, integers, characteristics, compounds, chemical moieties or groups described in conjunction with a particular aspect, embodiment or example of the invention are to be understood to be applicable to any other aspect, embodiment or example described herein unless incompatible therewith.

### DESCRIPTION OF VARIOUS EMBODIMENTS

### Glycoside/Glycosidic Compound

The terms “glycoside” or “glycosidic compound” as used herein are interchangeable and includes reference to any of the class of compounds that yield a sugar and an aglycone upon hydrolysis.

### Coumarin

The term “coumarin” as used herein includes reference to a compound comprising a chromenone ring. In one class of coumarin compounds, the chromenone ring is a chromen-2-one ring, while in another class the chromenone ring is a chromen-4-one ring. Many of the known coumarins are of the former class. Examples of coumarins of the latter class include quercetins and derivatives thereof.

### Hydrocarbyl

The term “hydrocarbyl” as used herein includes reference to a moiety consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19 or 20, carbon atoms. Examples of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g. cyclopropylmethyl); cycloalkyl (e.g. cyclopropyl, cyclobutyl, cyclopentyl or cyclohexyl); aryl (e.g. phenyl, naphthyl or fluorenyl) and the like.

### Alkyl

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, the alkyl moiety may have 1, 2, 3 or 4 carbon atoms.

### Alkenyl

The terms “alkenyl” and “C₂₋₆ alkenyl” as used herein include reference to a straight or branched chain alkyl moiety having 2, 3, 4, 5 or 6 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

### Alkynyl

The terms “alkynyl” and “C₂₋₆ alkynyl” as used herein include reference to a straight or branched chain alkyl moiety having 2, 3, 4, 5 or 6 carbon atoms and having, in addition, at least one triple bond. This term includes reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like.

### Cycloalkyl

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Cyclic Group

“Cyclic group” means a ring or ring system, which may be unsaturated or partially unsaturated but is usually saturated, typically containing 5 to 13 ring-forming atoms, for example a 5- or 6-membered ring. It includes carbocyclyl and heterocyclyl moeities.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered ring or ring system and, in particular, 5- or 6-membered rings, which may be saturated or unsaturated. A carbocyclic moiety is, for example, selected from cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like.

### Heterocyclyl

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, heterocyclyl includes a 3- to 10-membered ring or ring system and more particularly a 5- or 6-membered ring, which may be saturated or unsaturated.

A heterocyclic moiety is, for example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, pyrrolizidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

### Heteroaryl

The term “heteroaryl” as used herein includes reference to an aromatic heterocyclic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein includes reference to F, Cl, Br or I. In particular, a halogen may be F or Cl, of which F is more commonly used.

### Halogen Containing Moiety

The expression “halogen containing moiety” as used herein includes reference to a moiety comprising 1 to 30 plural valent atoms selected from carbon, nitrogen, oxygen and sulphur which moiety includes at least one halogen. The moiety may be hydrocarbyl for example C1-6 alkyl or C1-6 alkoxy, or carbocyclyl for example aryl.

### Substituted

The term “substituted” as used herein in reference to a moiety means that one or more, especially up to 5, more especially 1, 2 or 3, of the hydrogen atoms in said moiety are replaced independently of each other by the corresponding number of the described substituents. The term “optionally substituted” as used herein means substituted or un-substituted. It will, of course, be understood that substituents are only at positions where they are chemically possible, the person skilled in the art being able to decide (either experimentally or theoretically) without inappropriate effort whether a particular substitution is possible.

### Independently

Where two or more moieties are described as being “each independently” selected from a list of atoms or groups, this means that the moieties may be the same or different. The identity of each moiety is therefore independent of the identities of the one or more other moieties.

Embodiments of the invention are described below. Preferred features of each aspect of the invention are as for each of the other aspects mutatis mutandis. Moreover, it will be appreciated that the features specified in each embodiment may be combined with other specified features, to provide further embodiments.

### Compounds

The invention involves the use of coumarin compounds, in particular glycosidic coumarin compounds including derivatives of esculetin, umbelliferone, daphnetin, fraxetin or quercetin. Preferably the coumarin compounds are glycosidic coumarin compounds.

The glycosidic group may act as a substrate for an enzyme produced by the dermatophyte or any other microbe(s) present in proximity or as a co-infection.

In one embodiment, the invention provides compounds of the formula (I) or the formula (II):

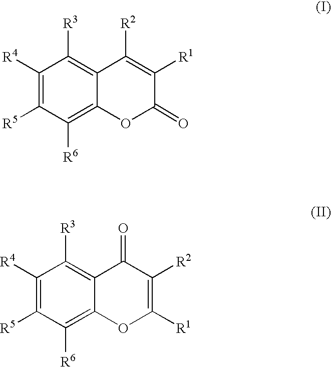

wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, R⁵ and R⁶ or R⁴, R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
and at least one of R¹, R², R³, R⁴, R⁵ and R⁶ or a said cyclic group comprises a glycosidic group;
   or a pharmaceutically acceptable salt or prodrug thereof.

The compound may comprise one or more (e.g. one or two) glycosidic groups, but usually comprises a single glycosidic group, typically at the 5-, 6-, 7- or 8-position of the coumarin ring. Thus, in one embodiment of formula (I), at least one of R³, R⁴, R⁵ and R⁶ comprises a glycosidic group. In a particular embodiment, at least one of R⁵, R⁶ and R⁷ comprises a glycosidic group. Of particular mention are compounds in which R⁵ or R⁶ comprises a glycosidic group. In the case of formula (II), a glycosidic group may especially be present at the 3-position, in which case R² comprises a glycosidic group.

Where other than a glycosidic group, R¹, R², R³, R⁴, R⁵ and R⁶ are often each independently selected from R⁷, —OR⁷, —C(O)R⁷ and —C(O)OR⁷ and R⁸, wherein:
R⁷ is independently selected from hydrogen; hydrocarbyl optionally substituted with 1, 2, 3, 4 or 5 R⁸; and —(CH₂)ₖ-heterocyclyl optionally substituted with 1, 2, 3, 4 or 5 R⁸;
R⁸ is independently selected from halogen, trifluoromethyl, cyano, nitro, oxo, ═NR⁹, —OR⁹, —C(O)R¹⁰, —C(O)N(R⁹)R¹⁰, —C(O)OR⁹, —OC(O)R⁹, —S(O)ₗR⁹, —S(O)ₗN(R⁹)R¹⁰, —N(R⁹)R¹⁰, —N(R⁹)N(R⁹)R¹⁰, —N(R⁹)C(O)R¹⁰ and —N(R⁹)S(O)ₗR¹⁰; and
R⁹ and R¹⁰ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3).

In particular, one or more of R¹, R², R³, R⁴, R⁵ and R⁶ may be each independently selected from hydrogen, halogen, trifluoromethyl, R⁷, —OR⁷, —C(O)R⁷ and —C(O)OR⁷, wherein R⁷ is hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy. In this regard, R⁷ is especially hydrogen or C₁₋₆ alkyl optionally substituted with 1, 2 or 3 substituents independently selected from halogen, cyano, amino, hydroxy and C₁₋₆ alkoxy. Thus, one or more of R¹, R², R³, R⁴, R⁵ and R⁶ may be each independently selected from hydrogen, halogen, trifluoromethyl, hydroxy, C₁₋₆ alkyl and C₁₋₆ alkoxy, wherein C₁₋₆ alkyl and C₁₋₆ alkoxy are optionally substituted with 1, 2, 3, 4 or 5 substituents independently selected from, for example, halogen (e.g. fluorine or chlorine), cyano, amino, hydroxy and C₁₋₆ alkoxy. In particular compounds, R¹ is hydrogen, hydroxy or acetyl; R² is hydrogen, hydroxy, methyl, methoxy, fluorine, chlorine or trifluoromethyl; and one of R³, R⁴, R⁵ and R⁶ comprises a glucoside group, and the others are each selected from hydrogen, hydroxy, methyl, methoxy, fluorine, chlorine or trifluoromethyl. Of particular mention are compounds in which one of R⁵ and R⁶ comprises a glycosidic group, and the other is hydroxy.

As mentioned above, one or more of R³ and R⁴, R⁴ and R⁵, or R⁵ and R⁶ taken together with the carbon atoms to which they are attached may form a cyclic group which is optionally substituted with halogen or a moiety comprising 1 to 30 plural valent atoms selected from carbon, nitrogen, oxygen and sulphur. The cyclic group may be a carbocyclyl (e.g. phenyl) or heterocyclyl (e.g. furanyl) group, either of which may be optionally substituted by, for example, one or more R⁷, —OR⁷, —C(O)R⁷ and —C(O)OR⁷. Alternatively, or additionally, a cyclic group so formed may comprise a glycosidic group, for example a sugar group.

In the case of compounds of formula (II), R¹ is often phenyl, optionally substituted with 1, 2, 3, 4 or 5 R⁸. This is especially in the case of compounds such as quercetins, in which R¹ is generally 1,2-dihydroxy-phen-4-yl. R² often comprises a glycosidic group, while R³ and R⁵ are often —OR⁷, especially hydroxy. In certain compounds of formula (II), R⁴ and R⁶ are each hydrogen.

The, or each, glycosidic group is generally a carbohydrate group, especially a monosaccharide, disaccharide or polysaccharide group, and may exist in various isomeric forms, for example α-D, α-L, β-D or β-L forms.

By way of illustration, a glycosidic group may be a group of one of the following formulae:

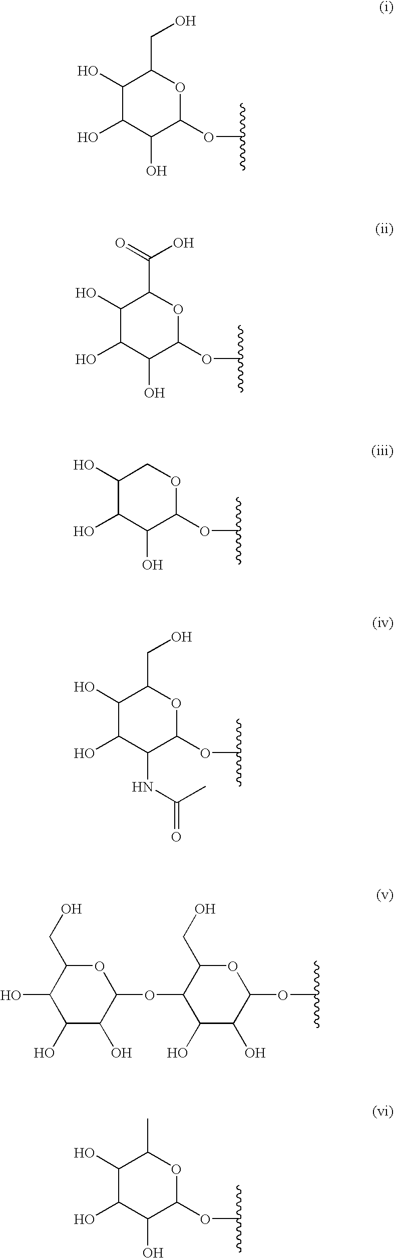

Exemplary glycosidic groups include glucopyranoside, galactopyranoside, mannopyranoside, fucopyranoside, arabinopyranoside, glucopyranoside, galactopyranoside, glucuronide, lactopyranoside, xylopyranoside, glucosaminide, galactosaminide, alloside, lyxoside, taloside, threoside, riboside, fructoside, rhamnoside and guloside groups. More particularly, the glycosidic group may be selected from α-D-glucopyranoside, α-D-galactopyranoside, α-D-mannopyranoside, α-L-fucopyranoside, α-L-arabinopyranoside, β-D-glucopyranoside, β-D-galactopyranoside, β-D-glucuronide, β-D-lactopyranoside, β-D-xylopyranoside, β-D-glucosaminide, β-D-galactosaminide, β-D-alloside, β-D-lyxoside, β-D-taloside, β-D-threoside, β-D-riboside, β-D-fructoside, β-D-rhamnoside and β-L-guloside groups.

Examples of compounds of the invention include those shown below. It will of course be appreciated that, where appropriate, each compound may be in the form of the free compound, an acid or base addition salt, or a prodrug.

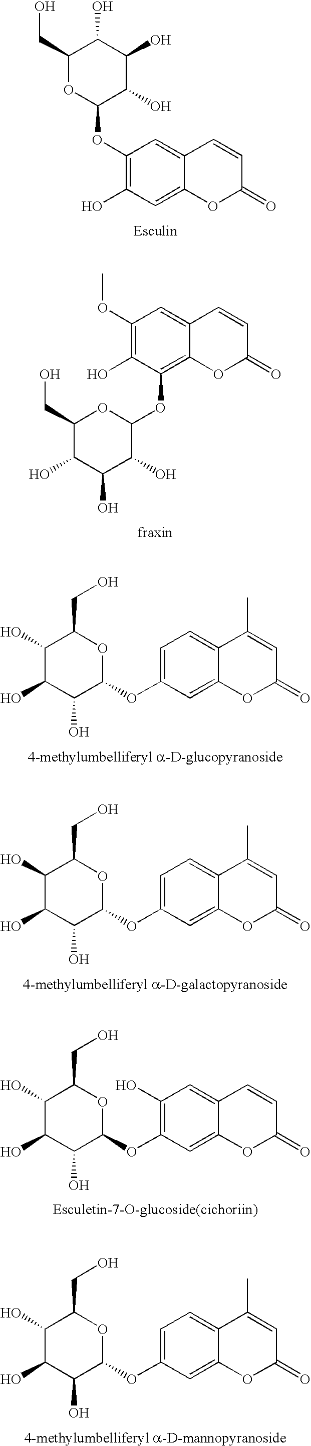

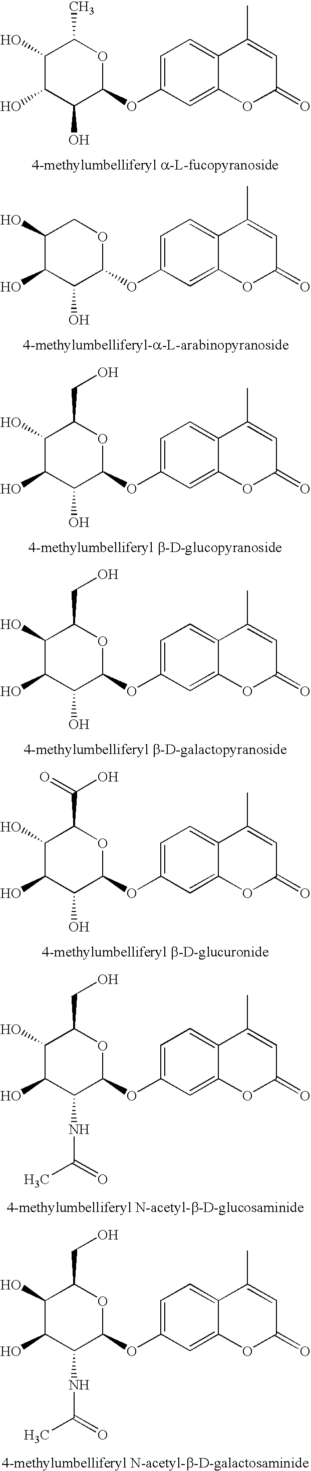

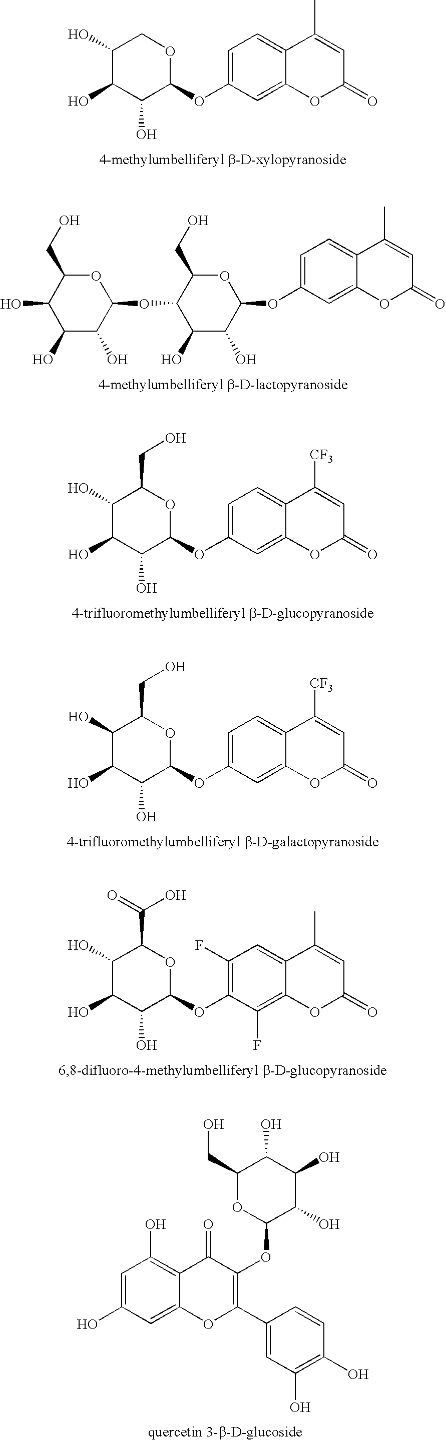

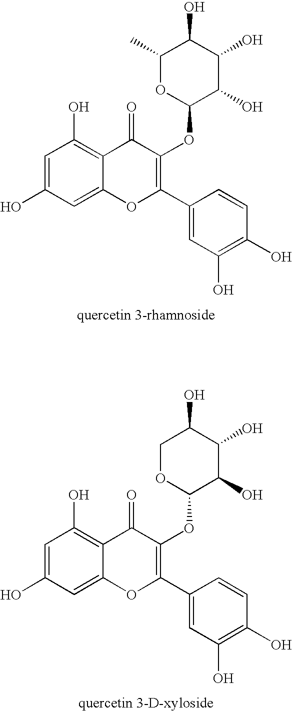

or a pharmaceutically acceptable salt or prodrug thereof.

The invention also involves the use of halogenated coumarin compounds, for example fluorinated compounds.

In one embodiment, the invention provides compounds of the formula (I) or the formula (II):

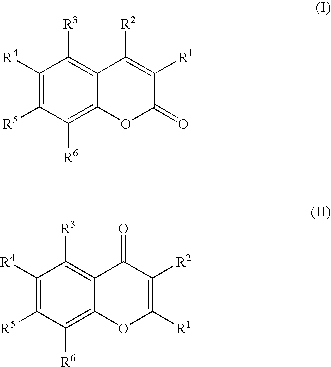

wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, R⁵ and R⁶ or R⁴, R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
wherein at least one of R¹, R², R³, R⁴, R⁵ and R⁶ or a said cyclic group comprises a halogen or halogen containing moiety;
   or a pharmaceutically acceptable salt or prodrug thereof.

The compound may comprise one or more (e.g. one or two) halogen or halogen containing moiety. Thus, in one embodiment of formula (I), at least one of R¹, R², R³, R⁴, R⁵ and R⁶ comprises a halogen or halogen containing moiety. Preferably, only one of R¹, R², R³, R⁴, R⁵ or R⁶ comprises a halogen or halogen containing moiety. Of particular mention are compounds in which R², R⁴ or R⁶ independently comprise a halogen or halogen containing moiety.

Where other than a halogen R¹, R², R³, R⁴, R⁵ and R⁶ may each be independently selected from R⁷, —OR⁷, —C(O)R⁷ and —C(O)OR⁷ and R⁸, wherein:
R⁷ is independently selected from hydrogen; hydrocarbyl optionally substituted with 1, 2, 3, 4 or 5 R⁸; and —(CH₂)ₖ-heterocyclyl optionally substituted with 1, 2, 3, 4 or 5 R⁸;
R⁸ is independently selected from halogen, trifluoromethyl, cyano, nitro, oxo, ═NR⁹, —OR⁹, —C(O)R¹⁰, —C(O)N(R⁹)R¹⁰, —C(O)OR⁹, —OC(O)R⁹, —S(O)ₗR⁹, —S(O)ₗN(R⁹)R¹⁰, —N(R⁹)R¹⁰, —N(R⁹)N(R⁹)R¹⁰, —N(R⁹)C(O)R¹⁰ and —N(R⁹)S(O)ₗR¹⁰; and
R⁹ and R¹⁰ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3).

In one embodiment the halogen is F.

In a further embodiment the halogen containing moiety is C1-6 alkyl (e.g. methyl) substituted with one, two or three halogens (for example F or Cl). The halogen containing moiety may be trifluoromethyl (e.g. 7-hydroxy-4-(trifluoromethyl)coumarin) or trichloromethyl.

Examples of compounds of the invention include those shown below. It will of course be appreciated that, where appropriate, each compound may be in the form of the free compound, an acid or base addition salt, or a prodrug:
6-Bromo-3-butyrylcoumarin
6-Bromocoumarin-3-carboxylic acid
6-Bromocoumarin-3-carboxylic acid
6,8-Dibromocoumarin-3-carboxylic acid
3-Chlorocoumarin
4-Chloro-3-nitrocoumarin
7-Amino-4-(trifluoromethyl)coumarin
7-Amino-4-(trifluoromethyl)coumarin
7-Hydroxy-4-(trifluoromethyl)coumarin
2,3,6,7-Tetrahydro-9-trifluoromethyl-1H,5H-quinolizino(9,1-gh)coumarin (Coumarin 153)
6-Bromo-3-(2,3-dichlorophenylcarbamoyl)-coumarin
7-Ethoxy-4-(trifluoromethyl)coumarin
7-Hydroxy-4-(trifluoromethyl)coumarin
7-Methoxy-4-(trifluoromethyl)coumarin
7-(Phenylacetamido)-4-(trifluoromethyl)coumarin
3-Acetyl-6-bromocoumarin
L-Alanine-7-amido-4-methylcoumarin trifluoroacetate
6-bromocoumarin
6-bromo-3-cyanocoumarin
6-bromo-3-cyano-4-methylcoumarin
6-bromo-4-hydroxycoumarin
6-bromomethyl-7-acetoxycoumarin
4-(bromomethyl)-6,7-dimethoxycoumarin
4-(bromomethyl)-7-methoxycoumarin
6-bromo-4-methyl-3-phenylcoumarin
3-butyryl-6,8-dibromocoumarin
6-chlorocoumarin
6-chloro-3-cyanocoumarin
6-chloro-3-cyano-4,7-dimethylcoumarin
6-chloro-3-cyano-4-methylcoumarin
6-chloro-3-cyano-4,7-dimethyl-3-phenylcoumarin
6-chloro-4-hydroxycoumarin
6-chloro-7-hydroxy-4-(methoxymethyl)coumarin
6-chloro-4-hydroxy-7-methylcoumarin
6-chloro-4-hydroxy-4-(trifluoromethyl)coumarin
6-chloro-4-methyl-7-phenylcoumarin
4-chloro-3-nitrocoumarin
6-(3-chloropropoxy)-4-methylcoumarin
3-cyano-6,8-dibromo-4-methylcoumarin
3-cyano-6,8-dichloro-4-methylcoumarin
3-cyano-6,7-dichloro-4-methylcoumarin
3-cyano-6-fluoro-4-methylcoumarin
6,8-dibromo-4-hydroxycoumarin
6,8-dibromocoumarin-3-carboxylic acid
6,8-dibromo-4-methyl-3-phenylcoumarin
6,7-dichloro-4-hydroxycoumarin
6,8-dichloro-4-hydroxycoumarin
6,7-dichloro-4-methyl-3-phenylcoumarin
6,8-dichloro-4-methyl-3-phenylcoumarin
ethyl 6,8-dibromocoumarin carboxylate
6-fluoro-4-hydroxycoumarin
6-fluoro-4-methyl-3-phenylcoumarin
7-hydroxy-4-(trifluoromethylphenyl)coumarin

As mentioned above, one or more of R³ and R⁴, R⁴ and R⁵, or R⁵ and R⁶ taken together with the carbon atoms to which they are attached may form a cyclic group which is optionally substituted with halogen or a halogen containing moiety comprising 1 to 30 plural valent atoms selected from carbon, nitrogen, oxygen and sulphur. The cyclic group may be a carbocyclyl (e.g. phenyl) or heterocyclyl (e.g. furanyl) group, either of which may be optionally substituted by, for example, one or more R⁷, —OR⁷, —C(O)R⁷ and —C(O)OR⁷. Alternatively or additionally, a cyclic group so formed may comprise a glycosidic group, for example a sugar group.

In one embodiment, the halogenated coumarin compound of the invention is a glycosidic compound.

Where appropriate, compounds of the invention may be in the form of pharmaceutically acceptable salts. The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

Pharmaceutically acceptable salts can be synthesized from the parent compound which contains a basic or acidic moiety by conventional chemical methods. Generally, such salts can be prepared by mixing the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure thus includes pharmaceutically-acceptable salts of the disclosed compounds wherein the parent compound is modified by making acid or base salts thereof. For example, the conventional non-toxic salts or the quaternary ammonium salts which are formed, e.g. from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs, therefore, include drugs having a functional group which has been transformed into a reversible derivative thereof. Typically, such prodrugs are transformed to the active drug by hydrolysis. Examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned oxidative activation (e.g. N- and O-dealkylation, oxidative deamination, N-oxidation or epoxidation) and reductive activation (e.g. azo reduction, sulfoxide reduction, disulfide reduction, bioreductive alkylation or nitro reduction).

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546), incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the disclosure may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the disclosure.

Compounds of the invention may exist in various isomeric or tautomeric forms. For example, in the case of compounds having a hydroxy group at the 7-position of the coumarin ring, the compounds may exist in either 6,7- or 2,6-form, as illustrated below in the case of esculin:

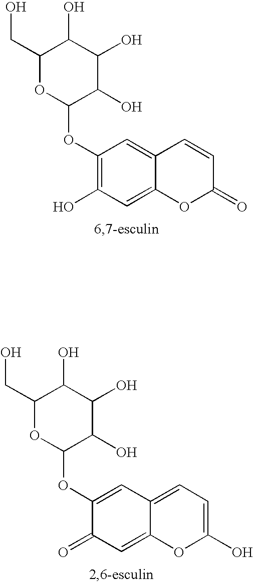

It will be appreciated that all such isomeric and tautomeric forms of the compounds are encompassed by the present invention.

The compounds of the disclosure may also contain one or more asymmetric carbon atoms and may therefore exhibit optical- and/or diastereo-isomerism. All diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively, the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the disclosure. Where a single enantiomer or diasteromer is disclosed, the disclosure also covers the other enantiomers or diastereomers, and also racemates; in this regard, particular reference is made to the specific compounds listed herein.

Geometric isomers may also exist in the compounds of the present disclosure. The present disclosure contemplates the various geometric isomers and mixtures thereof resulting from the arrangement of substituents around a carbon-carbon double bond and designates such isomers as of the Z or E configuration, wherein the term “Z” represents substituents on the same side of the carbon-carbon double bond and the term “E” represents substituents on opposite sides of the carbon-carbon double bond.

The disclosure therefore includes all variant forms of the defined compounds, for example any tautomer or any pharmaceutically acceptable salt, ester, acid or other variant of the defined compounds and their tautomers as well as substances which, upon administration, are capable of providing directly or indirectly a compound as defined above or providing a species which is capable of existing in equilibrium with such a compound.

### Synthesis

Compounds of the invention may occur naturally, be obtained commercially or synthesised using procedures well known to those skilled in the art, for example by a Perkin or Pechman synthesis.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

### Administration & Pharmaceutical Formulations

The compounds of the invention may be administered by any suitable route known to those skilled in the art. Depending upon the disorder and patient to be treated and the route of administration, the compositions may be administered at varying doses.

Typically, the compounds are administered topically. Exemplary, dosage forms for topical administration of a compound of this invention include powders, sprays and ointments. The active compound may be mixed under sterile conditions with a pharmaceutically acceptable carrier and any needed preservatives, buffers or propellants which may be required.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In the treatment, prevention, control, amelioration, or reduction of risk of conditions which require inhibition of fungal activity, an appropriate dosage level will generally be about 0.1 to about 100 mg/kg day The compounds may be administered in a regimen of 1 to 4 times per day. The dosage regime may be adjusted to provide the optimal therapeutic response.

### Use

As used herein, the term “dermatophytic infection” refers to an infection of the dermis or nails (fingernails, toenails, or, for non-human animals, hooves or claws) caused by a fungus. Such fungi include, but are not limited to, Trichophyton spp., Epidermophyton spp., and Microsporum spp.

Compounds of the invention may be useful in the therapy of a variety of topical nail infections in particular infections caused by dermatophytes. The infection may include a Tinea infection for example Tinea barbae (beard), Tinea capitis (head), Tinea corporis (body), Tinea cruris (groin), Tinea faciei (face), Tinea manuum (hand), Tinea pedis (foot) Tinea unguium (nail), Tinea (Pityriasis) versicolor, Tinea incognito or Tinea nigra. The infection may be derived from fungi of the genera Epidermophyton, Microsporum and Trichophyton spp. (e.g T. rubrum and T. interdigitale).

The dermatophytic infection may be an infection of the skin, lamina, stratum corneum, nails (fingernails and toenails) or hair. Of particular mention are dermatophytic infections caused by a dermatophyte of the genera Trichophyton, Epidermophyton or Microsporum. Exemplary dermatophytes include Epidermophyton floccosum, Microsporum canis, Microsporum audouinii, Microsporum gypseum, Microsporum nanum, Microsporum ferrugineum, Microsporum distortum, Microsporum fulvum, Trichophyton rubrum, Trichophyton mentagrophytes var. interdigitale, Trichophyton mentagrophytes var. nodulare, Trichophyton tonsurans, Trichophyton soudanese, Trichophyton violaceum, Trichophyton megnini, Trichophyton schoenlenii, Trichophyton gallinae, Trichophyton krajdenii, Trichophyton yaoundei, Trichophyton equinum, Trichophyton erinacei and Trichophyton verrucosum.

In a particular embodiment of the invention, the dermatophytic infection is onychomycosis. The term “onychomycosis” includes, but is not limited to, distal lateral subungual, superficial white, proximal white subungual, secondary dystrophic, primary dystrophic, endonyx, candidal (e.g. onycholysis & chronic mucocutaneous disease) types of onychomycosis and Tinea ungium.

Onychomycosis has been shown as a significant risk factor for more serious clinical complications, such as acute bacterial cellulitis of the arm/leg and other secondary bacterial infections, thus the present invention encompasses the treatment of these infections. Onychomycosis may be caused by a fungus from, but not limited to, the genus Trichophyton. For example, the fungus may be Trichophyton interdigitale or Trichophyton rubrum.

### BRIEF DESCRIPTION OF THE DRAWINGS

FIG. 1 is a histogram demonstrating the effect of the addition of β-glucosidase and esculin on the growth of Trichophyton rubrum NCPF118

FIG. 2 is a histogram demonstrating the effect of the addition of β-glucosidase and esculin on the growth of Trichophyton interdigitale NCPF335

FIG. 3 is a graph demonstrating the dose-dependent antifungal effect of esculetin versus T. rubrum NCPF118

FIG. 4 is a graph demonstrating the sensitivity of older cultures of T. rubrum NCPF118 and T. interdigitale NCPF335 to esculin.

FIG. 5 is a graph demonstrating the activity of coumarin glycosides (final concentration 5 mM) versus T. rubrum NCPF118.

FIG. 6 is a graph demonstrating the activity of a halogenated coumarin aglycone versus T. rubrum NCPF118.

The following Example illustrates the invention:

### Example

### Synthesis of Glycosidic Coumarins

Glycosidic coumarins were synthesised using a whole cell biocatalytic fermentation method based on that described in EK Lim et al., (2004), Biotech. Bioeng. 87 (636-637). Selected glycosytransferases were used to attach a glycoside to the following coumarins:

### Esculetin

4-(trifluoromethyl)coumarin

The glycoside was purified to 95% purity from the crude fermentation extract by HPLC methods known to the skilled person.

### Solubility of Coumarins—Aglycones Versus Respective Glycosides

Esculin, esculetin, 4-(trifluoromethyl) coumarin, daphnetin, fraxetin were purchased from Sigma-Aldrich Company Ltd.

The solubility of the aglycones was compared to the respective glycoside in RPMI 1640 media and in DMSO. The results are shown in Table 1:

TABLE 1
Solubility of selected coumarins and the corresponding glucosides

| | | Solubility in | DMSO | |
|---|---|---|---|---|
| | Compound | RPMI 1640¹ | Concentration | Theoretical |
| Compound | Type | (mM) | (% v/v) | Mean LogP² |
| Esculetin | Aglycone | 5 | 0.1 | +1.03 |
| (6,7 dihydroxycoumarin) | | | | |
| Esculin | Glucoside | 20 | 0 | −1.03 |
| (6,7 dihydroxycoumarin-6- | | | | |
| glucoside) | | | | |
| Cichorlin | Glucoside | 10 | 0³ | −1.06 |
| (6,7 dihydroxycoumarin-7- | | | | |
| glucoside) | | | | |
| 4-(trifluoromethyl)coumarin | Aglycone | 2 | 0.2 | +0.61 |
| 4-(trifluoromethyl)coumarin- | Glucoside | 10 | 0 | +0.33 |
| 7-glucoside | | | | |
| Fraxetin | Aglycone | 5 | 0.4 | +0.73 |
| (7,8 dihydroxy-6- | | | | |
| methoxycoumarin) | | | | |
| Fraxin | Glucoside | NA³ | NA³ | −1.10 |
| (Fraxetin-8-glucoside) | | | | |
| Daphnetin | Aglycone | 5 | 0.1 | +0.73 |
| (7,8-dihydroxycoumarin) | | | | |
| Daphnin | Glucoside | NA³ | NA³ | −1.13 |
| (Daphnetin-7-glucoside) | | | | |
| Daphnetin-8-glucoside | Glucoside | NA³ | NA³ | −1.01 |

¹pH = 7.0
²Calculated using ALOGPS program (Virtual Computational Chemistry Laboratory)
³Compound not available commercially

The results in Table 1 demonstrate that coumarin glucosides are significantly more soluble in RPMI1640 medium (pH7.0) and 0.0-0.4% (v/v) DMSO than the respective aglycones, and that untested coumarin glycosides are theoretically more soluble than the respective aglycones based on predicted partition coefficients (LogP). In the fields of organic and medicinal chemistry a partition coefficient or distribution coefficient is the ratio of concentrations of a compound in the two phases of a mixture of two immiscible solvents at equilibrium. Hence, these coefficients are a measure of differential solubility of the compound between these two solvents. A higher value indicates reduced aqueous solubility, whereas a lower number, including a negative value, indicates greater aqueous solubility.

### Anti-Fungal Activity of Coumarins Aglycones and Respective Glycosides

### Materials and Methods

Esculin, esculetin and a range of other coumarins and their glycosides were tested for their antifungal activity against Trichophyton rubrum, Trichophyton interdigitale and other fungi isolated from nails and skin.

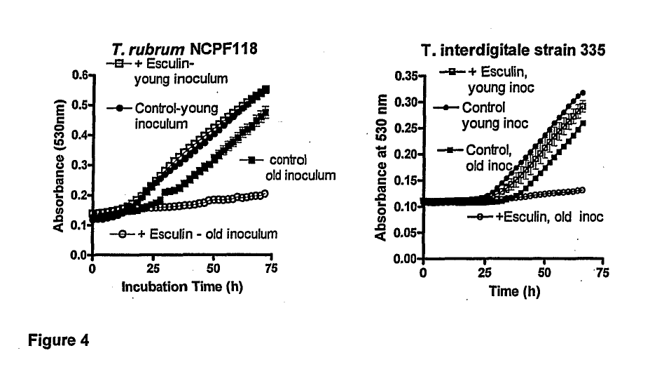

Suspensions of fungal conidia and hyphal fragments for use as experimental inocula were prepared by adding 3 ml of demineralised water or 0.15% (w/v) NaCl solution to a slope culture which had been incubated at 30° C. for 7 to 10 days and probing the surface of the culture with a sterile plastic transfer pipette. In the case of clinical isolates demonstrating slow or poor growth the culture period was extended for up to 18 days to allow sufficient growth for the preparation of a dense spore suspension. The resulting suspension was filtered through a double layer of sterile surgical gauze; if necessary a further volume of up to 3 ml of water or 0.15% (w/v) NaCl was used to wash through any spores or small hyphal fragments held on the gauze. 100 μl of this suspension, adjusted to an optical density between 0.14 and 0.16 at 530 nm (corresponding to between 0.5 and 1.5×10⁶ propagules/ml) was added to the wells in sterile 96-well microtitre plates to which 100 μl of 2× strength RPMI 1640 media (a chemically-defined nutrient medium widely used in tissue culture and antimicrobial resistance testing) had been added. Inocula prepared in this way were used in all of the experiments; additionally, for some experiments with esculin shown in FIG. 4, the results obtained with standard inocula prepared as described above (labelled “young inoculum” in FIG. 4), were compared with the results obtained in similar tests using inocula which had been cultured for up to 9 weeks (labelled “old inoculum” in FIG. 4). As appropriate, coumarins were pre-dissolved in 2× strength RPMI 1640 medium before addition to the plate. Experiments with the yeasts Candida albicans and Candida krusei were set up in the same way, but in these cases, filtration of the cell suspensions was not necessary.

Esculin dissolves readily in 2×RPMI 1640 solution. Solubilisation of other compounds required the presence of DMSO. Esculetin, daphnetin, 4-(trifluoromethyl) coumarin, 4-(trifluoromethyl)-coumarin-7-0-glucose and cichoriin were dissolved in DMSO at 1000× final test concentration; when this solution was diluted to the concentration used in the experiments using 2× strength RPMI 1640 medium, the final concentration of DMSO was 0.05% which alone had no effect on the fungi when tested in several experiments performed. Fraxetin required a final concentration of 0.2% DMSO, but this too had no effect on the growth of the fungi when tested in separate experiments.

A series of dilutions of the coumarin solutions was prepared on the plates by mixing known volumes of the coumarin stock solutions in the wells with fresh volumes of 2×RPMI 1640 medium. Each concentration of coumarin was studied in triplicate wells. For observations on Trichophyton rubrum NCPF118 and Trichophyton interdigitale NCPF335, obtained from the National Collection of Pathogenic Fungi (Bristol), the plates were incubated in a Bio-Tek Powerwave XS scanning plate reader at 30° C., and fungal growth within the plates was measured at intervals of 2 h for up to 72 h or 96 h as specified in the text by recording the absorbance (optical density) of the wells at 530 nm. Observations on Candida albicans and Candida krusei were made in the same way, but the incubation temperature was 37° C. and the incubation time was 48 h. For observations with clinical fungal isolates obtained from the collection of M. Monod (Lausanne, Switzerland), the procedure was as described above except that in response to the relatively slow growth of these isolates, incubations were performed in a static incubator at 30° C. and the plates were read manually in a Bio-Tek Powerwave XS scanning plate reader at intervals of 24 h for up to 168 h, being returned to the incubator after each reading. Malassezia furfur grows poorly in the system described above, so for this organism, a standard inoculum of M. furfur was mixed with an equal volume of a solution of esculetin and the mixture was plated onto Petri dishes containing Sabouraud's agar medium. Growth was monitored visually during incubation at 37° C. for up to 96 h and was compared with growth in the absence of the coumarins.

### Results

Table 2 demonstrates that a number of the coumarin aglycones tested were inhibitory to Trichophyton spp. reference strains at concentrations ranging between 0.25 and 1 mM

TABLE 2

| Strain | Coumarin | MIC (mM) |
|---|---|---|
| T. rubrum NCPF118 | Daphnetin | 0.5 |
| T. rubrum NCPF118 | Fraxetin | 0.25 |
| T. rubrum NCPF118 | 7-hydroxy-4 | 1.0 |
| | trifluoromethyl | |
| | coumarin | |
| T. rubrum NCPF118 | 4 methyl esculetin | 1.0 |
| T. interdigitale NCPF335 | Daphnetin | 0.25 |
| T. interdigitale NCPF335 | Fraxetin | 0.5 |

The sensitivity of clinical isolates of dermatophytes to esculetin is demonstrated in Table 3

TABLE 3

| | No of strains | MIC esculetin, | Sensitive (S) or |
|---|---|---|---|
| Species | tested | range in mM | Resistant (R) |
| Trichophyton rubrum | 48 | 0.54 ± 0.37 | S |
| T. rubrum strain 1072 | 3 | ≧2 | R |
| T. mentagrophytes | 27 | 1.1 ± 0.5 | S |
| T. mentagrophytes var | 1 | ≧2 | R |
| interdigitale strain 386 | | | |
| T. tonsurans | 2 | 0.25 ± 0.18 | S |
| T. soudanense | 2 | 0.31 ± 0.27 | S |
| Microsporum canis | 4 | 1.0 ± 0.58 | S |
| M. gypseum | 1 | ≧2 | R |
| Total | 88 | | 83S/5R |

The sensitivity of other fungal isolates of pathogenic fungi to esculetin is demonstrated in Table 4

TABLE 4

| | | | Sensitive (S) |
|---|---|---|---|
| | No of strains | MIC Esculetin, | or |
| Species | tested | (mM) | Resistant (R) |
| Fusarium spp. | 15 | ≧2 | R |
| Aspergillus fumigatus | 1 | ≧2 | R |
| Scopulariopsis brevicaulis | 4 | ≧2 | R |
| Alternaria spp. | 1 | 1 | S |
| Curvularia spp. | 1 | ≧2 | R |
| Total | 22 | | 1S/21R |

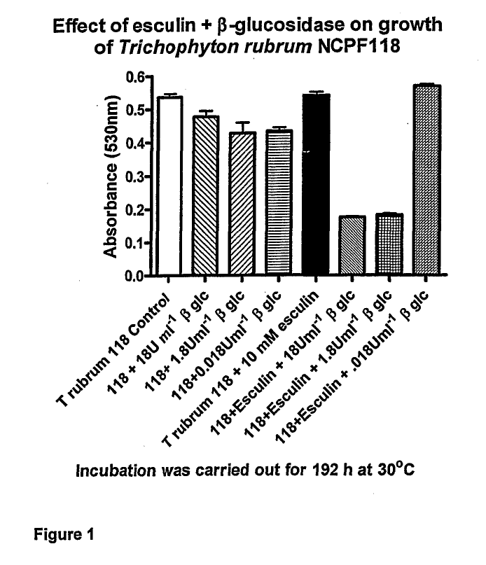

FIG. 1 demonstrates that the addition of β-glucosidase and esculin to Trichophyton rubrum NCPF118 significantly inhibits fungal growth

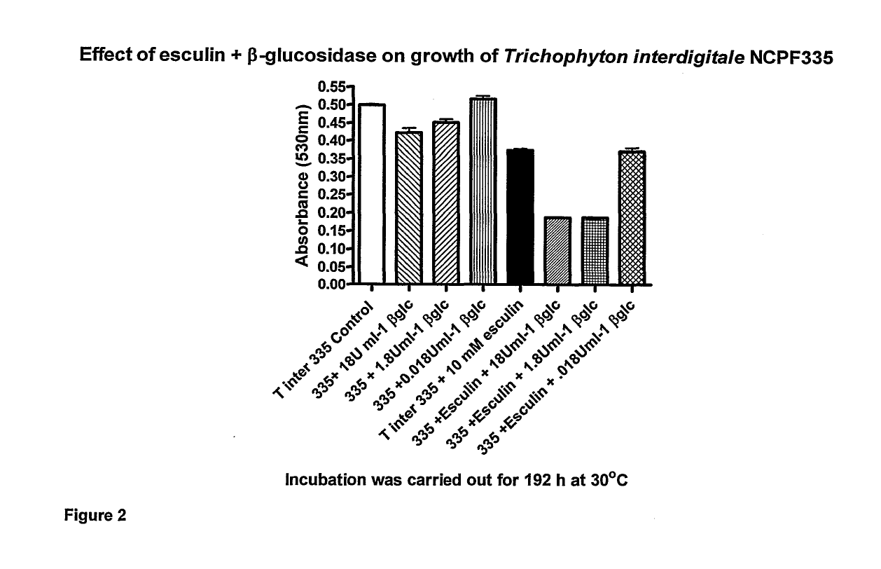

FIG. 2 demonstrates that the addition of β-glucosidase and esculin to Trichophyton interdigitale NCPF335 significantly inhibits fungal growth

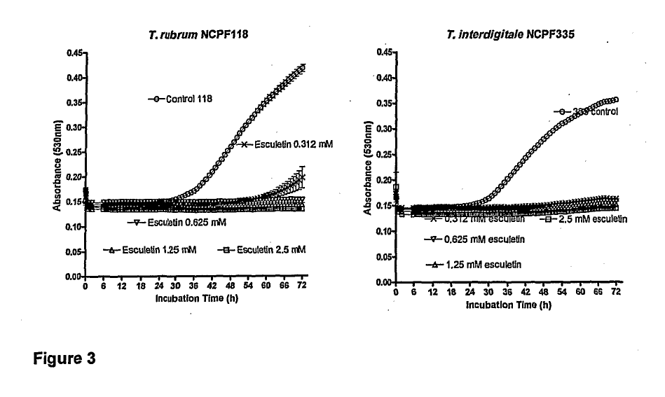

FIG. 3 demonstrates the dose-dependent antifungal effect of esculetin versus T. rubrum NCPF118

FIG. 4 demonstrates that older cultures of T. rubrum NCPF118 and T. interdigitale NCPF335 are more sensitive to esculin, due to increased production of β-glucosidase in older cultures

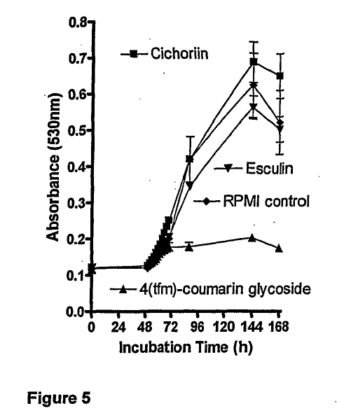

FIG. 5 demonstrates the activity of coumarin glycosides versus T. rubrum NCPF118.

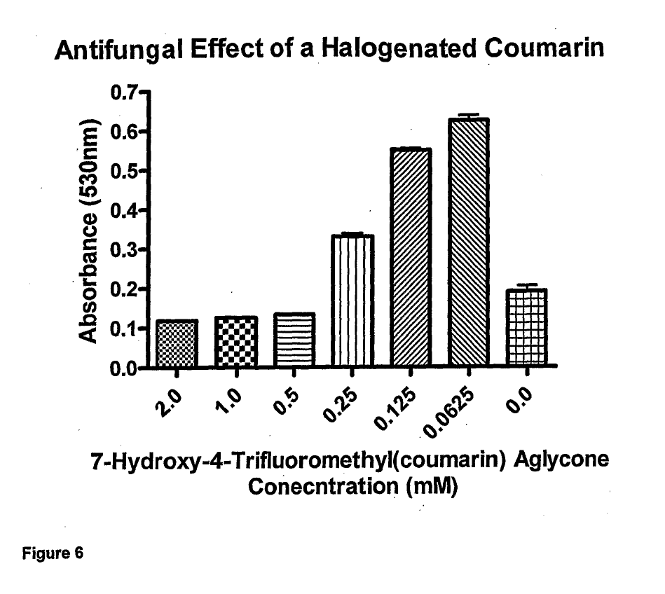

FIG. 6 demonstrates that despite only being soluble at low concentrations, the aglycone of 7-hydroxy-4-(trifluoromethyl)coumarin is significantly antifungal versus T. rubrum NCPF118 at concentrations of 1-2 mM.

1. A coumarin compound or a pharmaceutically acceptable salt or prodrug thereof, for use in the treatment, prevention or delay of progression of a dermatophytic infection in a patient.

2. The compound according to claim 1 which is a glycosidic coumarin compound.

3. The compound according to claim 1, wherein the infection is caused by a dermatophyte of the genera Trichophyton, Epidermophyton or Microsporum.

4. The compound according to claim 3, wherein the dermatophtye is selected from the group consisting of Epidermophyton floccosum, Microsporum canis, Microsporum audouinii, Microsporum gypseum, Microsporum nanum, Microsporum ferrugineum, Microsporum distortum, Microsporum fulvum, Trichophyton rubrum, Trichophyton mentagrophytes var. interdigitale, Trichophyton mentagrophytes var. nodulare, Trichophyton tonsurans, Trichophyton Soudanese, Trichophyton violaceum, Trichophyton megnini, Trichophyton schoenlenii, Trichophyton gallinae, Trichophyton krajdenii, Trichophyton yaoundei, Trichophyton equinum, Trichophyton erinacei and Trichophyton verrucosum.

5. The compound according to claim 4, wherein the dermatophyte is Trichophyton rubrum.

6. The compound according to claim 1, wherein the infection is onychomycosis.

7. The compound according to claim 1, which is of the formula (I) or the formula (II): 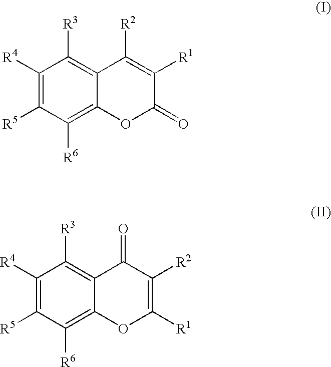
wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, or R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
and at least one of R¹, R², R³, R⁴, R⁵ and R⁶ or a said cyclic group comprises a glycosidic group;
or a pharmaceutically acceptable salt or prodrug thereof.

8. The compound according to claim 7, wherein R², R⁴, R⁵ or R⁶ comprises a glycosidic group.

9. The compound according to claim 1, wherein the compound comprises a glycosidic group which is a monosaccharide, disaccharide or polysaccharide group.

10. The compound according to claim 9, wherein the glycosidic group is selected from glucopyranoside, galactopyranoside, mannopyranoside, fucopyranoside, arabinopyranoside, glucopyranoside, galactopyranoside, glucuronide, lactopyranoside, xylopyranoside, glucosaminide, galactosaminide, alloside, lyxoside, taloside, threoside, riboside, fructoside, rhamnoside and guloside groups.

11. The compound according to claim 9, wherein the glycosidic group selected from α-D-glucopyranoside, α-D-galactopyranoside, α-D-mannopyranoside, α-L-fucopyranoside, α-L-arabinopyranoside, β-D-glucopyranoside, β-D-galactopyranoside, β-D-glucuronide, β-D-lactopyranoside, β-D-xylopyranoside, β-D-glucosaminide, β-D-galactosaminide, β-D-alloside, β-D-lyxoside, β-D-taloside, β-D-threoside, β-D-riboside, β-D-fructoside, β-D-rhamnoside and β-L-guloside groups.

12. The compound according to claim 2, which is selected from: 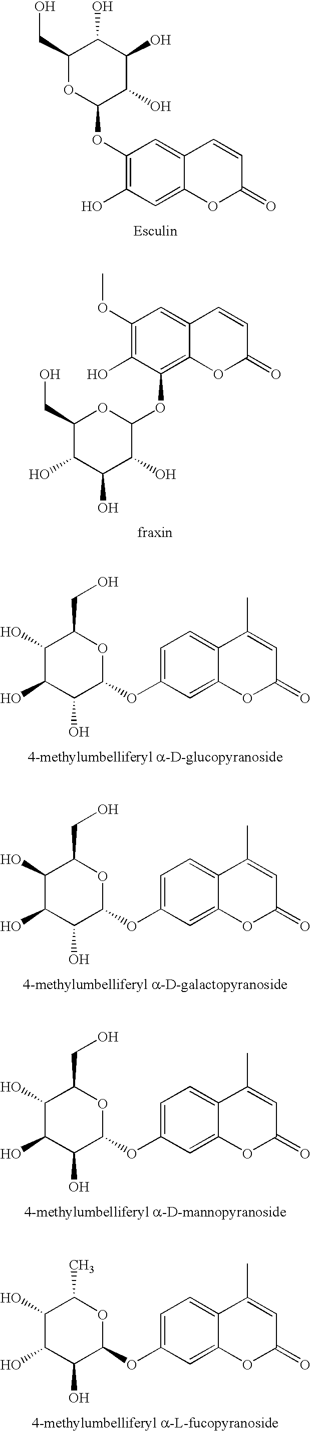 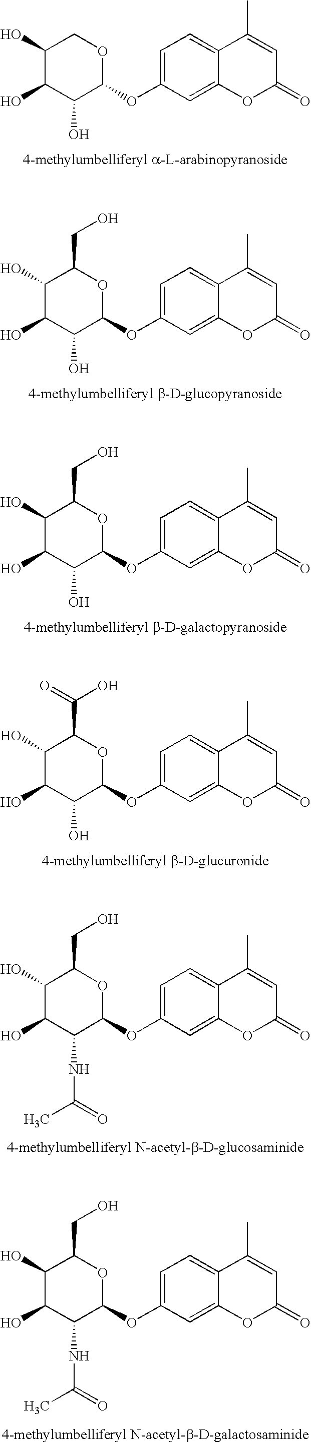 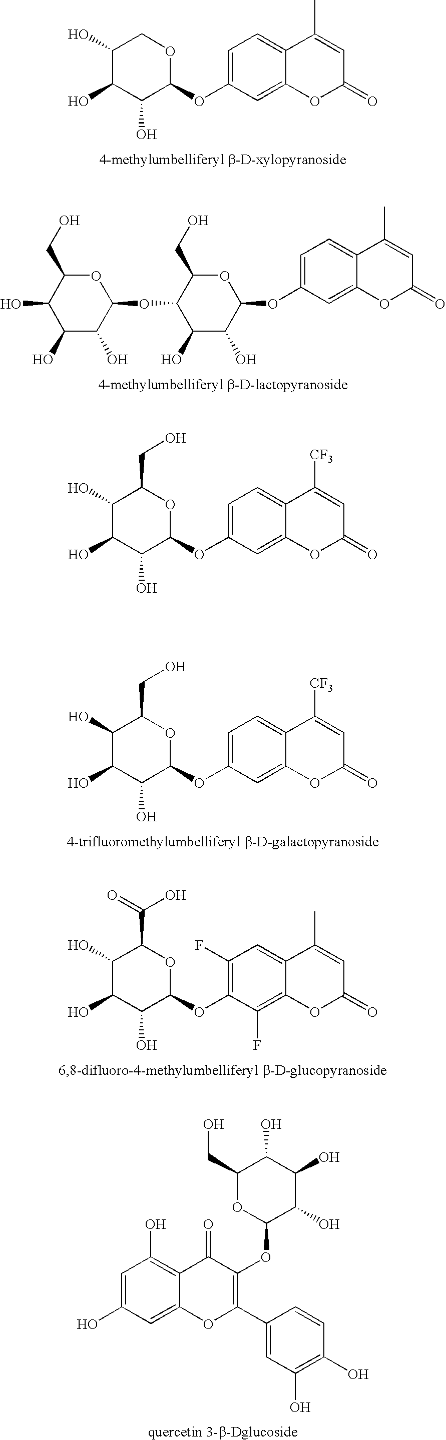 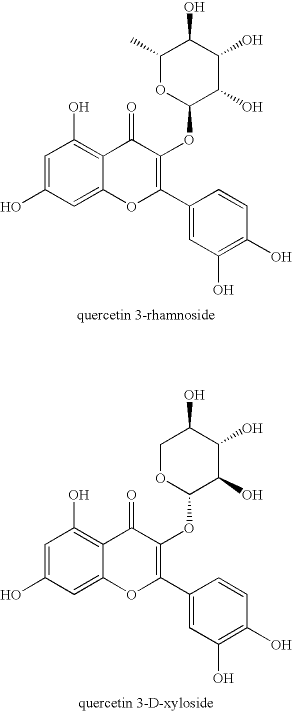
or a pharmaceutically acceptable salt or prodrug thereof.

13. The compound according to claim 12, which is esculin or a prodrug thereof.

14. The compound according to claim 1 which is a halogenated coumarin compound.

15. A pharmaceutical formulation comprising a coumarin compound or a pharmaceutically acceptable salt or prodrug thereof, for use in the treatment, prevention or delay of progression of a dermatophytic infection in a patient.

16. The formulation according to claim 15, wherein the coumarin compound is a glycosidic coumarin compound.

17. The formulation according to claim 15, wherein the infection is caused by a dermatophyte of the genera Trichophyton, Epidermophyton or Microsporum.

18. The formulation according to claim 16, wherein the compound is of the formula (I) or the formula (II): 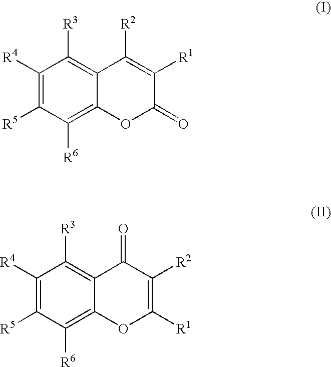
wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, or R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
and at least one of R¹, R², R³, R⁴, R⁵ and R⁶ or a said cyclic group comprises a glycosidic group;
or a pharmaceutically acceptable salt or prodrug thereof.

19. The formulation according to claim 15, which further comprises a pharmaceutically acceptable adjuvant, diluent or carrier.

20. Use of a coumarin compound or a pharmaceutically acceptable salt or prodrug thereof, for the manufacture of a medicament for the treatment, prevention or delay of progression of a dermatophyte infection.

21. Use according to claim 20, wherein the coumarin compound is a glycosidic coumarin compound.

22. Use according to claim 21, wherein the compound has the formula (I) or the formula (II): 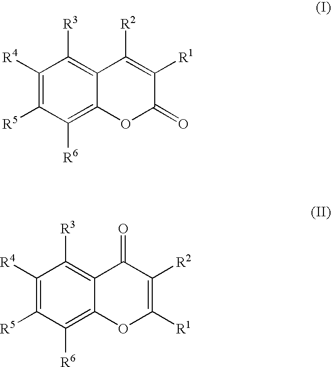
wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, or R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
and at least one of R¹, R², R³, R⁴, R⁵ and R⁶ or a said cyclic group comprises a glycosidic group;
or a pharmaceutically acceptable salt or prodrug thereof.

23. Use according to claim 20, wherein the coumarin compound is a halogenated coumarin compound.

24. Use according to claim 20, wherein the infection is caused by a dermatophyte of the genera Trichophyton, Epidermophyton or Microsporum.

25. A method for the treatment, prevention or delay of progression of a dermatophytic infection, which comprises administering to a patient a therapeutically effective amount of a coumarin compound or a pharmaceutically acceptable salt or prodrug thereof.

26. The method according to claim 25 wherein the compound is a glycosidic coumarin compound.

27. The method according to claim 25, wherein the compound is a halogenated coumarin compound.

28. The method according to claim 26, wherein the compound has the formula (I) or the formula (II): 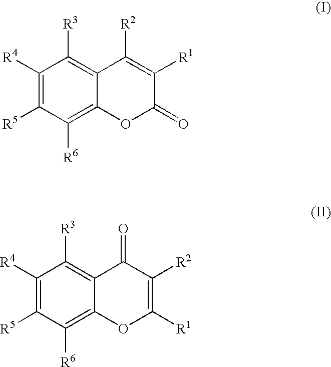
wherein
R¹, R², R³, R⁴, R⁵ and R⁶ are each independently hydrogen, halogen or a moiety comprising 1 to 30 plural valent atoms selected from carbon, nitrogen, oxygen and sulphur;
or R³ and R⁴, R⁴ and R⁵, or R⁵ and R⁶ may be taken together with the carbon atoms to which they are attached to form a cyclic group which is optionally substituted with halogen or a moiety comprising 1 to 30 plural valent atoms selected from carbon, nitrogen, oxygen and sulphur;
and at least one of R¹, R², R³, R⁴, R⁵ and R⁶ or a said cyclic group comprises a glycosidic group;
or a pharmaceutically acceptable salt or prodrug thereof.

29. The method according to claim 25, wherein the infection is caused by a dermatophyte of the genera Trichophyton, Epidermophyton or Microsporum.
